(19) United States
(12) Patent Application Publication
(10) Pub. No.: US 2011/0257474 A1
(43) Pub. Date: Oct. 20, 2011

(54) APPLICATOR GLOVE

(76) Inventor: RHEYNARD WENDEL HOWARD, Missouri, TX (US)

(21) Appl. No.: 13/086,581

(22) Filed: Apr. 14, 2011

Related U.S. Application Data
(60) Provisional application No. 61/323,996, filed on Apr. 14, 2010.

(51) Int. Cl. A61F 5/00; A41D 19/00

(52) U.S. Cl. 600/38; 2/163

(57) ABSTRACT

A glove used for the application of colognes, perfumes and flavors to the exterior or interior of the human body. The glove has a flexible sleeve including one or more finger sections, a thumb section, and protuberances attached on at least one of the finger sections or the thumb section.

### CROSS-REFERENCE TO RELATED APPLICATIONS

This application claims the benefit of priority to U.S. Provisional Application No. 61/323,996 filed Apr. 14, 2010, the contents of which are incorporated herein by reference.

### BACKGROUND OF THE INVENTION

The present invention generally relates to a flexible glove, and more particularly to a glove used for the application of colognes, perfumes and flavors externally and/or internally to the human body.

Sexually transmitted diseases (STDs) are infections that are transmitted between humans by means of human sexual behavior, including vaginal intercourse, oral sex, and anal sex. Those that are sexually active with different partners have high chances of contracting STDs such as herpes, genital warts, gonorrhea, syphilis, or human immunodeficiency virus (HIV). It is public domain that manual stimulation increases the chances of obtaining the climax during a sexual act. Unfortunately, open sores on the hands or fingers can often lead to the transmission of STDs as a result of hand fondling other's genitals and inserting fingers into bodily openings.

As can be seen, there is a need for a device that allows manual stimulation of a partner during sex and at the same time protect the user from STDs.

### SUMMARY OF THE INVENTION

In one aspect of the present invention, a glove has a flexible sleeve including one or more finger sections, a thumb section, and protuberances attached on at least one of the finger sections or the thumb section.

In another aspect of the present invention, a glove includes a flexible sleeve including one or more finger sections, a thumb section, and protuberances attached on at least one of the finger sections or the thumb section, and a connector adapted to operatively connect a light source or a vibration device to the glove.

These and other features, aspects and advantages of the present invention will become better understood with reference to the following drawings, description and claims.

### BRIEF DESCRIPTION OF THE DRAWINGS

FIG. 1 illustrates a perspective front view of an applicator glove according to an exemplary embodiment of the present invention;

FIG. 2 illustrates a perspective front view of an applicator glove according to a second exemplary embodiment of the present invention;

FIG. 3 illustrates a perspective front view of an applicator glove according to a third exemplary embodiment of the present invention;

FIG. 4 illustrates a perspective front view of an applicator glove according to a fourth exemplary embodiment of the present invention; and

FIG. 5 illustrates a perspective front view of an applicator glove according to a fifth exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, embodiments of the present invention generally provide a glove used for the application of colognes, perfumes and flavors to the exterior or interior of the human body.

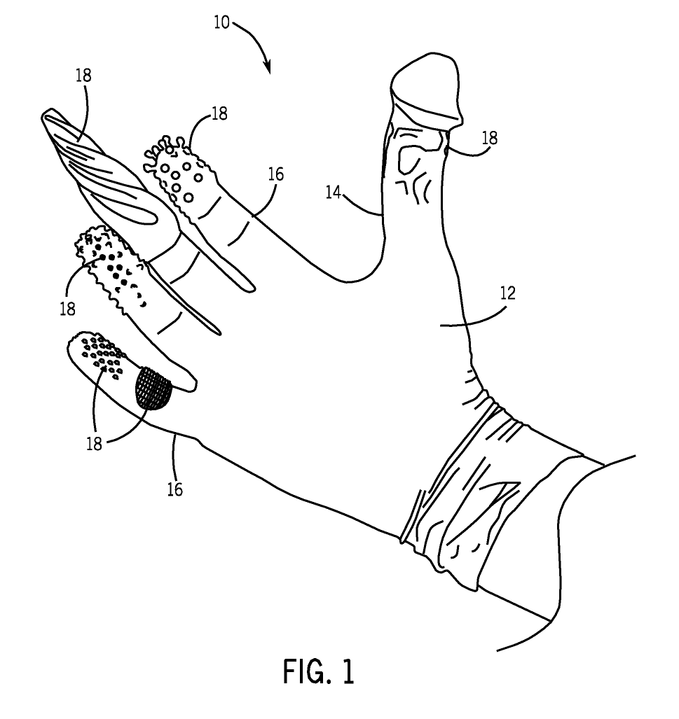

FIGS. 1-5 illustrate different exemplary embodiments of a glove 10 according to the present invention. The glove 10 may include a flexible sleeve 12 including one or more finger sections 16, a thumb section 14, and protuberances 18 attached on at least one of the finger sections 16 or the thumb section 14.

The flexible sleeve 12 may have a shape that resembles the shape of a right or left human hand. The size of the flexible sleeve 12 may depend on the size of the hand of the user. The flexible sleeve 12 may be made of a very thin, highly flexible elastic material. In one embodiment, the material of the glove 10 may be safe for internal insertion into the human body. In some embodiments, the flexible sleeve 12 may be made of rubber, plastic, polyvinyl chloride (PVC), or latex. The flexible sleeve 12 may be made by dipping a mold into latex, injection molding, or blow molding. In some embodiments, the flexible sleeve 12 may be made by pouring or extruding hardened latex or plastic material to form a cast.

Each finger section 16 may have a shape that resembles the shape of one of the human fingers.

The protuberances 18 may have a shape or texture that may enhance tactile sensation. In some embodiments, the shape of the protuberances 18 may resemble bumps, ribs, swirls, knobs, dots, ridges, jelly knobs, spheres, g-spot hooks, suction cups, knobby clitoral ticklers, jelly tips, vibrating tips, dildos, small balls, bubbles, plugs, balls, cones, spikes, curves tips, or raised bumps.

The protuberances 18 may partially or completely cover each finger section 16 and/or thumb section 14. In some embodiments, the protuberances 18 may be placed in one side of the finger section 16 and/or thumb section 14. In some embodiments, the protuberances 18 may be placed in both sides of the finger section 16 and/or thumb section 14. In some embodiments, the protuberances 18 may be placed in the tip of the finger section 16 and/or thumb section 14. In some embodiments, at least one finger section 16 and/or thumb section may be free of the protuberances 18.

The shape of the protuberances 18 may be the same or different for each finger section 16 and/or thumb section 14.

The protuberances 18 may be embedded to the flexible sleeve 12 during the manufacturing process. In some embodiments, the protuberances may be removable so that they may be interchanged.

The protuberances 18 may be created by forming depressions in the wall either during molding of the applicator or subsequently.

In one embodiment, the protuberances 18 may be impregnated with liquids or gels during the manufacturing process. The liquids or gels may be lubricating agents, perfumes, colognes, antiseptic agents, oils, flavoring agents, desensitizing agents, sexual stimulation or enhancing formulations, creams, medications, or microbiological agents. In some embodiments, the user may add the liquid or gel at the time of use.

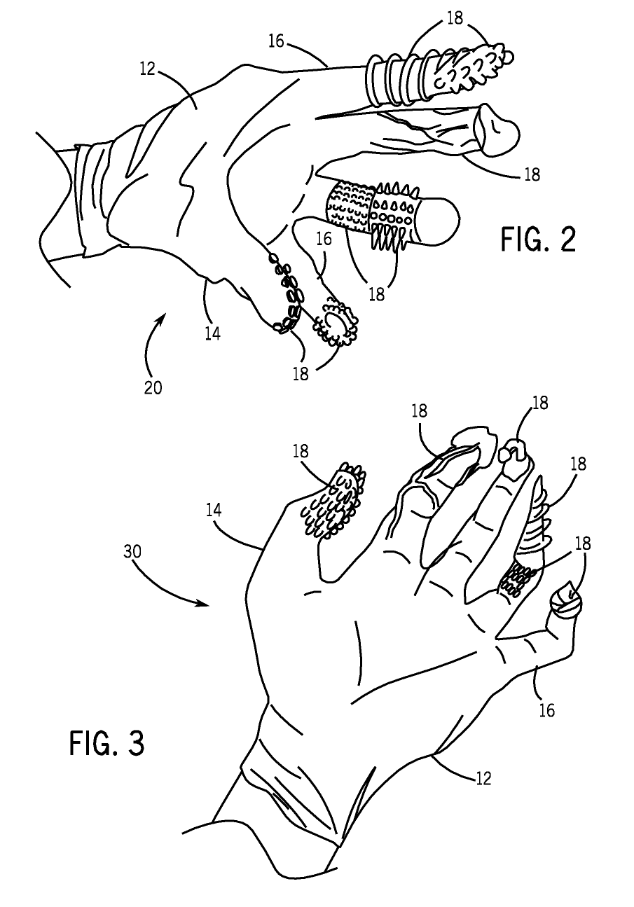

FIG. 2 illustrates an exemplary embodiment of a glove 20 according to an exemplary embodiment of the present invention including protuberances having a different set of shapes.

FIG. 3 illustrates an exemplary embodiment of a glove 30 according to an exemplary embodiment of the present invention including protuberances having another set of shapes.

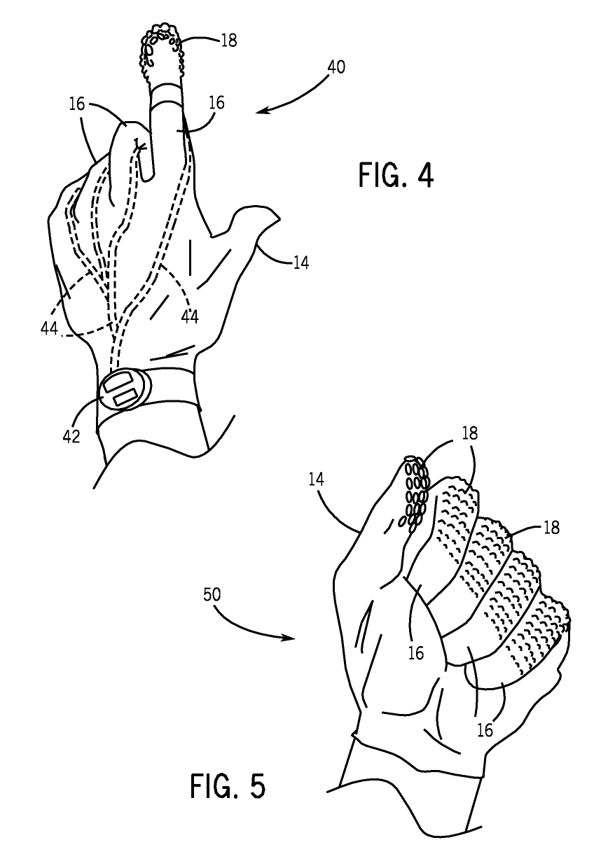

FIG. 4 illustrates an exemplary embodiment of a glove 40 according to an exemplary embodiment of the present invention. A connector 42 may be attached to the flexible sleeve 12. The connector 42 may allow connecting a light source (not shown) or a vibration device (not shown) to the glove 10. Electric cords 44 may help to connect the connector 42 with the light source (not shown) or a vibration device (not shown).

FIG. 5 illustrates an exemplary embodiment of a glove 50 according to an exemplary embodiment of the present invention including protuberances having another set of shapes.

The glove 10 may be custom made according to the requirements of the user.

The glove 10 may keep the hands of the user safe from body fluids and smells, massage the internal or external parts of a human body, and at the same time provide sexual and health gratification to the recipient.

It should be understood, of course, that the foregoing relates to exemplary embodiments of the invention and that modifications may be made without departing from the spirit and scope of the invention as set forth in the following claims.

What is claimed is:

1. A glove comprising:
a flexible sleeve including one or more finger sections, a thumb section, and protuberances having a shape attached on at least one of the finger sections or the thumb section.

2. The glove according to claim 1, wherein the shape of each protuberance is different on each finger section or thumb section.

3. The glove according to claim 1, wherein the shape of each protuberance is the same on each finger section or thumb section.

4. The glove according to claim 1, wherein the shape of the protuberances includes bumps, ribs, swirls, knobs, dots, ridges, jelly knobs, spheres, g-spot hooks, suction cups, knobby clitoral ticklers, jelly tips, vibrating tips, dildos, or combination thereof.

5. The glove according to claim 1, wherein the protuberances are embedded in the flexible sleeve.

6. The glove according to claim 1, wherein the protuberances are removable attached to the flexible sleeve.

7. The glove according to claim 1, wherein the glove is impregnated during a manufacturing process with lubricating agents, perfumes, colognes, antiseptic agents, oils, flavoring agents, desensitizing agents, sexual stimulation or enhancing formulations, creams, medications, or microbiological agents.

8. A glove comprising:
a flexible sleeve including one or more finger sections, a thumb section, and protuberances having a shape attached on at least one of the finger sections or the thumb section; and
a connector adapted to operatively connect a light source or a vibration device to the glove.

9. The glove according to claim 8, wherein the shape of each protuberance is different on each finger section or thumb section.

10. The glove according to claim 8, wherein the shape of each protuberance is the same on each finger section or thumb section.
